(19) United States
(12) Patent Application Publication
(10) Pub. No.: US 2003/0045874 A1
(43) Pub. Date: Mar. 6, 2003

(54) Transverse connector assembly for spine fixation system

(76) Inventor: James C. Thomas JR., Las Vegas, NV (US)

Correspondence Address: CHRISTIE, PARKER & HALE, LLP, 350 WEST COLORADO BOULEVARD, SUITE 500, PASADENA, CA, 91105, US

(21) Appl. No.: 09/944,681

(22) Filed: Aug. 31, 2001

(51) Int. Cl. A61B 17/70

(52) U.S. Cl. 606/61

(57) ABSTRACT

A screw-clamp assembly is useful for connecting a pair of spine rods in a spinal fixation system. The screw-clamp assembly includes an upper half-clamp, a lower half-clamp, an anchor screw, and a nut for clamping the half-clamps together. The upper and lower half-clamps mate to define a pair of openings for receiving the spine rods. The anchor screw is placed through an aperture in the lower half-clamp, an aperture in the upper half-clamp, and is threaded into a nut. By tightening the nut, the half-clamps are drawn together to securely grip the spine rods. A pair of screw clamp assemblies are arranged with perpendicular openings to be combined with a transverse rod to form a transverse connector assembly for bridging a pair of generally parallel spine rods.

### BACKGROUND OF THE INVENTION

This invention relates to an implantable spinal fixation system for the surgical treatment of spinal disorders. More specifically, it relates to a transverse connector assembly for attaching the spine rods of spinal fixation systems to one another.

Various types of spinal column disorders are known and include scoliosis (abnormal lateral curvature of the spine), kyphosis (abnormal backward curvature of the spine), lordosis (abnormal forward curvature of the spine), spondylolisthesis (forward displacement of a lumbar vertebra) and other disorders, such as ruptured or slipped discs, broken or fractured vertebrae, and the like. Patients who suffer from such conditions usually experience extreme and debilitating pain. A technique known as spinal fixation uses surgical implants which mechanically immobilize areas of the spine causing the eventual fusion of the treated vertebrae. Such techniques have been used effectively to treat the above-described conditions and, in most cases, to bring to the patient relief from pain.

One particular technique for spinal fixation includes the immobilization of the spine by the use of a pair of spine rods that run generally parallel to the spine. In practicing this technique, bone screws are first fastened to the pedicles of the appropriate vertebrae or to the sacrum and act as the anchor points for the spine rods. The bone screws are generally placed two per vertebra, one at each pedicle on either side of the spinous process. Clamp assemblies join the spine rods to the screws. The spine rods are generally custom-bent to achieve the desired curvature of the spinal column. Examples of such spinal fixation devices can be found in U.S. Pat. Nos. 4,653,481; 5,330,473; 5,520,687; and 5,030,220, which are incorporated herein by reference. For some applications, rather than using bone screws, lamina hooks can be fastened to a spine rod to connect the rod to a vertebra for distraction or compression.

It has been found that when a pair of spine rods are fastened in parallel on either side of the spinous process, the assembly can be significantly strengthened by using at least one additional rod to horizontally bridge the pair of spine rods.

### SUMMARY OF THE INVENTION

A spinal fixation system has a transverse connector assembly for connecting a first spine rod to a second spine rod. The assembly includes two screw-clamp assemblies and a transverse rod. The clamp assemblies include upper and lower half-clamps that each have two channels that mate to define a pair of openings. The first opening receives the first spine rod and the second opening receives the second spine rod. The assembly also comprises an anchor screw that extends through a first aperture defined by the lower half-clamp and a second aperture defined by the upper half-clamp and threads into a sleeve nut. By tightening the sleeve nut, the two half-clamps can be drawn together to securely grip the rods.

In a preferred embodiment of the screw-clamp assembly, the openings are arranged generally perpendicular to one another to provide a screw-clamp assembly that connects a transverse rod perpendicularly to a spine rod. A pair of such screw-clamp assemblies is used with a transverse rod to form a transverse connector assembly between a generally parallel pair of spine rods.

### BRIEF DESCRIPTION OF THE DRAWINGS

Features, aspects and advantages of the invention will be more fully understood when considered with respect to the following detailed description, appended claims, and accompanying drawings where:

FIG. 1 is a perspective posterior view of a spinal fixation system including a transverse connector assembly of the present invention installed on a portion of the spinal column;

FIG. 2 is an exploded elevation view, partly in section, of the second clamp assembly portion of a transverse cross brace of the present invention;

FIG. 3 is a top view of the lower half-clamp of the screw-clamp assembly showing the channels; and

FIG. 4 is a top view of the upper half-clamp of the screw-clamp assembly showing the channels and aperture for the anchor screw and sleeve nut.

FIG. 5 is a perspective view of the screw-clamp assembly showing a crimp securing the assembly.

### DETAILED DESCRIPTION OF THE INVENTION

The present invention relates to a transverse connector assembly for use in connecting spine rods, the spine rods being part of a spinal fixation system useful for treating various spinal disorders. The transverse connector assembly is useful to provide additional lateral support to a pair of generally parallel spine rods fastened to the same general portions of the spine, but on opposite sides of the spinous process.

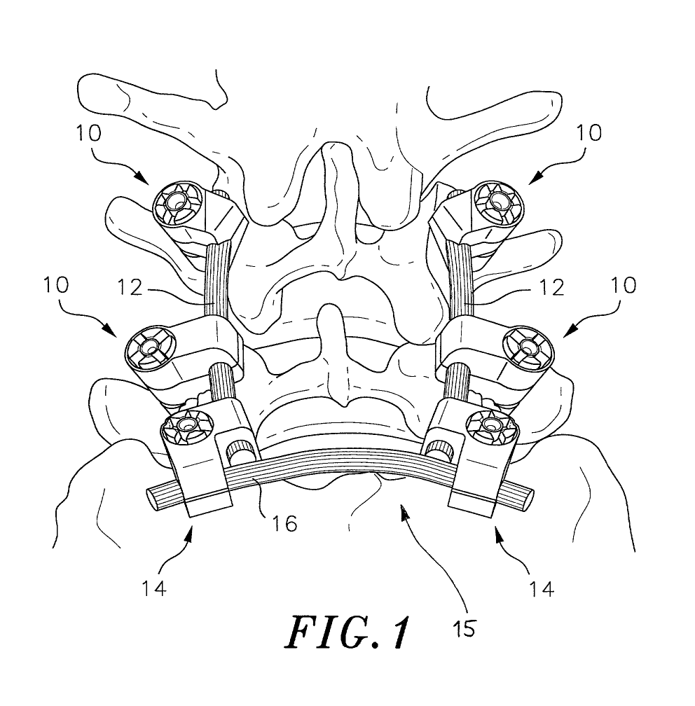

FIG. 1 illustrates an embodiment of a branch connector assembly in place on a spine. Spinal fixation systems generally use rods, pedicle or anchor screws, nuts, spacers, and clamps to provide the necessary support for immobilizing a spine. Conventional screw-and-clamp or “screw-clamp” assemblies 10 are fastened to the pedicles of one or more vertebrae. For this embodiment, two generally parallel spine rods 12 are fastened to the screw-clamp assemblies to provide the vertical support for the assembly. The details of such general assemblies are known, for example, as described in U.S. Pat. No. 5,520,687, which is hereby incorporated by reference.

This embodiment further includes a transverse connector 15 of the present invention. A transverse rod 16 is held by a pair of half-clamps in each screw-clamp assemblies 14 at the sacrum. Each screw-clamp assembly further fastens to the lower ends of a spine rod. Because both a spine rod 12 and a transverse rod 16 are held by each screw clamp assembly 14, the system has fewer parts, simplifying the surgical installation procedure.

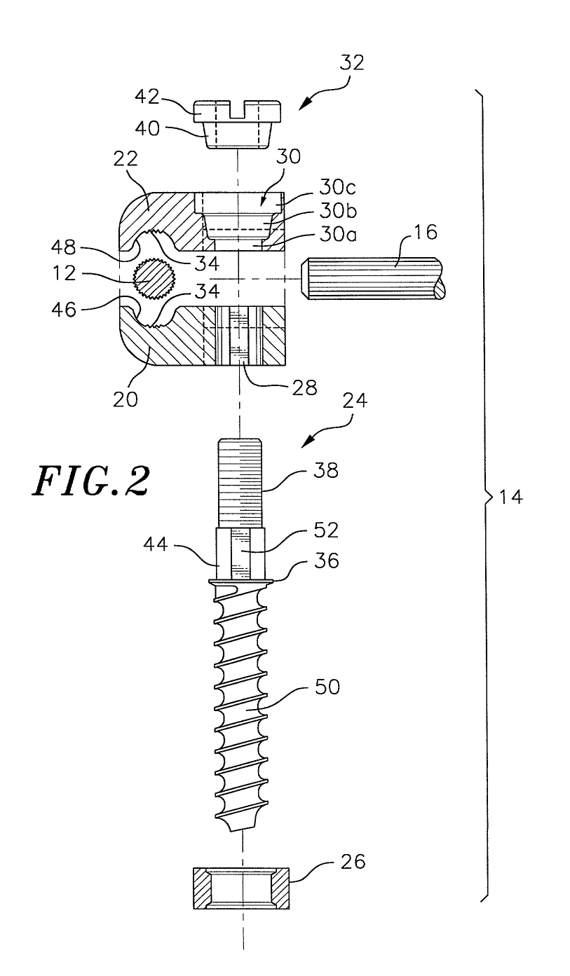

An exploded view of the screw-clamp assembly 14 is shown in FIG. 2. In the embodiment shown in FIG. 2, each screw-clamp assembly 14 includes a lower half-clamp 20 and an upper half-clamp 22 which mate to define a pair of openings for receiving a spine rod 12 and a transverse rod 16. The lower half-clamp 20 and the upper half-clamp 22 are positioned on top of an anchor screw 24. A sleeve nut 32 is threaded onto the anchor screw 24 to press the upper and lower half-clamps toward one another, clamping around the spine rod 12 and transverse rod 16. Optionally, a spacer 26 is positioned below the lower half-clamp 20 on the anchor screw 24.

The anchor screw 24, includes a lower coarsely-threaded end 50 for attachment to a bone such as a pedicle or the sacrum. Opposite the coarsely-threaded end 50 of the anchor screw 24 is a more finely threaded end 38 onto which the sleeve nut 32 is threaded. Between the two threaded ends are a shoulder 36 adjacent the coarsely threaded end and a neck 44 between the shoulder and the finely threaded end. The screw-clamp assembly 14 is assembled such that the bottom surface of the lower half-clamp 20 rests upon the top of the shoulder 36 of the anchor screw 24. This arrangement helps to keep the clamp assembly from pressing into the sacrum or vertebrae when the device is assembled. In the preferred embodiment as illustrated in FIG. 2, a spacer 26 is interposed between the bottom surface of the shoulder 36 of the anchor screw 24 and the bone. Spacers can be provided of different heights to permit the half-clamps to be spaced an appropriate distance from the patient's spine.

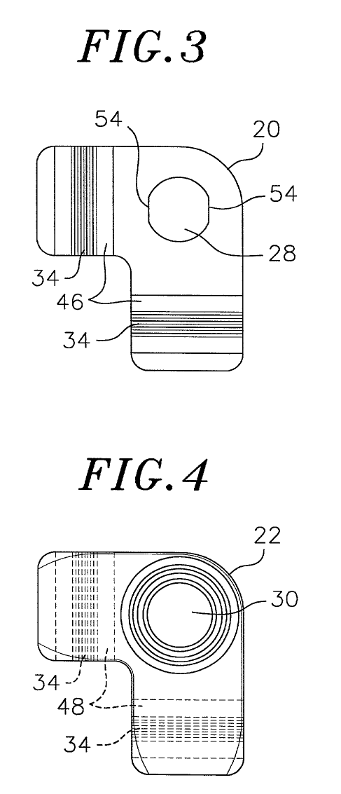

FIG. 3 illustrates a top view of a lower half-clamp 20 of the screw-clamp assembly 14. The lower half-clamp 20 is provided with an aperture 28 for receiving the anchor screw 24. In the embodiment shown in FIG. 3, aperture 28 defines an inner wall provided with opposing inner flat surfaces 54 which mate with optional outer flat surfaces 52 provided on the neck of the anchor screw. The lower half-clamp 20 further defines two channels 46 to each receive a rod. The channels 46 are generally perpendicular to each other so that one will accommodate a generally vertical spine rod 12 and the outer will accommodate a generally horizontal transverse rod 16. Each channel 46 is serrated along its length with teeth 34.

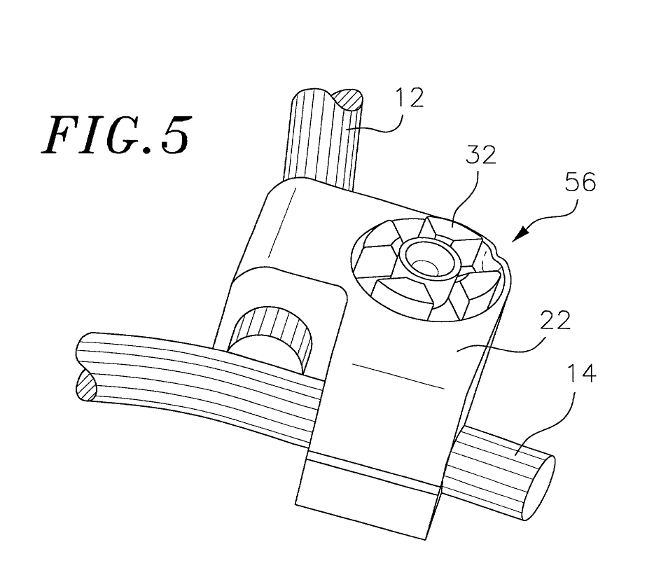

FIG. 4 illustrates a top view of the structure of the upper half-clamp 22 of the screw-clamp assembly 14. The upper half-clamp 22 is provided with an aperture 30 for receiving the anchor screw 24. The upper portion of the aperture 30 is recessed to accommodate the sleeve nut 32. The recess is formed by a stepped diameter for the aperture 30, as shown in section in FIG. 2. A lower portion 30a of the aperture 30 is narrow to accommodate only the threaded portion 38 of the anchor screw 24. An intermediate portion 30b of the aperture 30 has an intermediate diameter which is wider than the lower portion 30a to accommodate the small-diameter portion 40 of the sleeve nut 32. An upper portion 30c of the aperture 30 has a large diameter which is wider than the intermediate portion 30b to accommodate the large-diameter portion 42 of the sleeve nut 32. The upper half-clamp 22 defines a plastically deformable thin wall between the aperture 30 and the closest outer surface of the upper half-clamp 22 that may be crimped to secure the sleeve nut 32.

Two channels 48 are provided on the upper half-clamp 22 to each receive a rod. The channels 48 are generally perpendicular to each other so that one will accommodate a generally vertical spine rod 12 and the other will accommodate a generally horizontal transverse rod 16. Each channel 48 is serrated along its length with teeth 34 for improved purchase, especially when used with serrated rods. When the screw-clamp assembly 14 is assembled, the channels 48 of the upper half-clamp 22 and the channels 46 of the lower half-clamp 20 mate and together define openings to accommodate the spine rod 12 and the transverse rod 16.

The sleeve nut 32 is of the same general design as the sleeve nut described in U.S. Pat. No. 5,520,687. The sleeve nut 32 has a threaded aperture which mates with the finely threaded portion 38 of the anchor screw 24. The exterior of the sleeve nut 32 has a stepped diameter to mate with the recessed aperture 30 of the upper half-clamp 22. At the lower end of the sleeve nut, a small-diameter portion 40 of the sleeve nut is at its smallest and is sized such that the sleeve nut will fit into the intermediate portion 30b of the recessed aperture 30 of the upper half-clamp 22. At the upper end of the sleeve nut, adjacent the small-diameter portion 40, is a large-diameter portion of the sleeve nut 42. A fillet radius is located at the juncture of the small and large-diameter portion of the sleeve nut. The large-diameter portion 42 is sized so that it will fit into the upper portion 30c of the recessed aperture 30 of the upper half-clamp 22, thus pressing the upper half-clamp 22 and the lower half-clamp toward one another and against the shoulder when the sleeve nut 32 is threaded onto the anchor screw 24. The stepped interior diameter of the aperture 30 of the upper half-clamp 22 allows a distribution of the force conferred by the sleeve nut 32 on the upper half-clamp 22 over a larger area.

The top face of the sleeve nut 32 includes four radial notches, placed at equal distances from each other. The notches permit the use of a suitable driver for tightening the sleeve nut 32 onto the anchor screw and into the upper half-clamp 22. Preferably the sleeve nut is tightened to about 100 in. lb. of torque. In a preferred embodiment, the driver comprises a mechanism for holding the sleeve nut so that the surgeon can more easily attach the sleeve nut to the anchor screw. Optionally, as shown in FIG. 5, once the transverse connector has been assembled, a crimp 56 is placed in the thin wall of the upper half-clamp 22 where there is a radial notch in the associated sleeve nut 32 to lock the sleeve nut 32 in place and prevent it from loosening accidentally.

While the spine rods used in the particular spinal fixation device illustrated are generally of the same diameter, this need not always be the case. For example, different diameter spine rods 12 or transverse rods 16 may be desirable. By configuring a screw-clamp assembly with differently sized channels, a screw-clamp assembly of this invention can be used to securely connect the different sized rods.

In the preferred embodiment, spine rods 12 and transverse rod 14 are longitudinally serrated and the channels of the upper and lower half-clamps include teeth 34 for gripping the serrations. The teeth 34 run the width of the channels in a direction generally parallel to the direction of the rod that is to be received by the channel. Once assembled, the serrations of the rods and the teeth 34 of the upper and lower half-clamps cooperate to provide a firm grip between the rods and the screw-clamp assembly 14, virtually eliminating any later slippage of the device.

Tests have shown that a cross brace assembly using serrated rods and toothed transverse clamps provides exceptional resistance to rotational forces. The addition of a transverse connector assembly of this design to a spinal fixation system can significantly increase the rotational stiffness over a system without such a transverse connector assembly. As proper treatment of a diseased or injured spine requires its complete immobilization, a transverse connector assembly of this invention can dramatically improve the effectiveness of a spinal fixation system. The transverse rod 16 is generally installed after the rest of the vertical assembly is in place. The transverse rod 16 is cut to length and bent as necessary so that its ends simultaneously seat in each opening of each screw-clamp assembly 14. The upper half-clamps 22 can then be placed over the anchor screws 24 and locked into place with the sleeve nuts 32.

The screw clamp assemblies of the present invention are generally installed as follows. Once the appropriate portion of a patient's spine has been exposed, holes are drilled and tapped into the sacrum and pedicles where the anchor screws 24 are to be placed. Depending on the position of an anchor screw 24, an appropriate spacer 26 is selected and placed upon the anchor screw 24 from the lower coarsely-threaded end 50. The anchor screw 24 with the spacer 26 is then threaded into the tap hole. The flat surfaces 52 of the neck portions 44 of the anchor screws 24 can be used to drive the anchor screws 24 into place. Alternatively, the spacer 26 may be omitted. A lower half-clamp 20 is positioned over the anchor screw 24 and lowered to contact the shoulder 36 of the anchor screw 24. A spine rod 12 and a transverse rod 16 are cut to an appropriate length and then bent to fit into the appropriate channels of the screw clamp assemblies 14 and vertical clamp assemblies 10. An upper half-clamp 22 is positioned on each lower half-clamp 20 such that the channels 48 are over the spine rods 12 and transverse rod 16. A sleeve nut 32 is threaded onto the threaded portion 38 of each anchor screw 24 and tightened to an appropriate torque. In order to lock each sleeve nut, a crimp is placed in the thin wall of the associated upper half-clamp. Because the spine and transverse rods are bent, the assembly of this invention has considerable advantages over devices of the prior art which employ straight spine rods because it is not necessary to align the anchor screws with each other perfectly during the difficult surgical procedure.

The transverse connector assembly of this invention generally has a low profile compared to prior art devices for connecting pairs of spine rods. This is because the devices of this invention keep the two vertical spine rods and transverse rod to be joined in the same general plane while providing a firm connection between them. A particularly low profile design is further achieved by seating the head of the sleeve nut 32 within the recessed aperture 30 of the upper half-clamp 22. The head of the sleeve nut 32 includes perpendicular slots in its upper surface which allow the use of a suitable tool to drive the thread of the sleeve nut 32 into engagement with the threaded portion 38 of the anchor screw 24.

The transverse connector assembly as described above is generally for use in the lumbar region and is most preferably installed in the sacrum. However, the assembly can also be installed in a pedicle or vertebra for use in the thoracic or cervical regions of the spine. For some regions of the spine, the transverse connector may need to be scaled down in size somewhat. Similarly, while a transverse rod of the same diameter as the spine rods has been illustrated, the channels of the clamps can be modified to accommodate a transverse rod of a different diameter. The channels can also be modified to accommodate a differently shaped rod such as one with an elliptical or polygonal transverse cross section. In some instances, two or more transverse connectors may be used. For example, one may be placed at the sacrum as shown with a second in the pedicles of a vertebrae such as the second or third lumbar vertebrae.

The vertical and screw-clamp assemblies of the present invention are preferably made of an alloy capable of resisting corrosion when installed in a human body. It has been found that 316 stainless steel which has been electropolished and passivated to resist corrosion works well. Other metal alloys, such as alloys of titanium also work well.

The present invention is not to be limited to the specific designs shown which are merely illustrative. Various and numerous other arrangements may be devised by one skilled in the art without departing from the spirit and scope of this invention. The scope of the invention is defined in the following claims.

What is claimed is:

1. A screw-clamp assembly for use in spinal support fixation systems comprising:
an anchor screw having a first threaded end for extending above a bone and a second threaded end for placement in the bone;
a clamping assembly mounted on the first end of the anchor screw and comprising a lower half-clamp and an upper-half-clamp each with a recessed aperture defined therein to receive the anchor screw and each with a first and a second channel to receive a rod wherein the first and second channels are generally perpendicular to each other; and
a sleeve nut with threads adapted to thread onto the first end of the anchor screw and to be received within the recessed aperture of the upper-half-clamp for securing the lower-half-clamp and upper-half-clamp to the first end of the anchor screw.

2. The screw-clamp assembly of claim 1 wherein the sleeve nut has a stepped external diameter and the aperture in the upper-half-clamp has a stepped diameter.

3. The screw-clamp assembly of claim 1 wherein the channels are serrated along their lengths.

4. The screw-clamp assembly of claim 1 further comprising a shoulder located between the first and second threaded ends of the anchor screw and a spacer to be placed around the second end of the anchor screw to be positioned between the bone and the shoulder.

5. The screw-clamp assembly of claim 1 wherein the lower half-clamp and the upper half-clamp mate to define a first opening comprising the first channel of the lower half-clamp and the first channel of the upper half-clamp and a second opening comprising the second channel of the lower half-clamp and the second channel of the upper half-clamp.

6. The screw-clamp assembly of claim 5 further comprising a first rod placed in the first opening and a second rod placed in the second opening.

7. The screw-clamp assembly of claim 5 wherein the upper half-clamp defines a deformable wall which can be crimped to secure the sleeve nut.

8. The screw-clamp of claim 1 wherein the anchor screw, lower half-clamp, upper half-clamp, and sleeve nut are each fabricated from stainless steel or titanium.

9. A low-profile screw-clamp assembly for use in spinal support fixation systems comprising:
an anchor screw having a first threaded end for extending above a bone and a second threaded end for placement in the bone;
a clamping assembly mounted on the first end of the anchor screw, comprising a lower-half-clamp and an upper-half-clamp wherein each half-clamp has a first channel, a second channel generally perpendicular to the first channel, and an aperture defined therein; and
a sleeve nut with threads adapted to thread onto the first end of the anchor screw and to be received within the aperture of the upper half-clamp for securing the lower-half-clamp and upper-half-clamp to the first end of the anchor screw wherein the sleeve nut and the first end of the anchor screw do not extend beyond the upper surface of the upper-half-clamp distal to the bone when the clamping assembly is affixed to the anchor screw to thereby provide a smooth, single-plane surface, distal to the bone into which the spinal support fixation systems are implanted.

10. The screw-clamp assembly of claim 9 wherein the sleeve nut has a stepped external diameter and the aperture in the upper half-clamp has a stepped diameter.

11. The low-profile screw-clamp assembly of claim 9 wherein the channels are serrated along their lengths.

12. The screw-clamp assembly of claim 9 further comprising a shoulder located between the first and second threaded ends of the anchor screw and a spacer to be placed around the second end of the anchor screw to be positioned between the bone and the shoulder.

13. The screw-clamp assembly of claim 9 wherein the lower half-clamp and the upper half-clamp mate to define a first opening comprising the first channel of the lower half-clamp and the first channel of the upper half-clamp and a second opening comprising the second channel of the lower half-clamp and the second channel of the upper half-clamp.

14. The screw-clamp assembly of claim 13 further comprising a first rod placed in the first opening and a second rod placed in the second opening.

15. The screw-clamp assembly of claim 13 wherein the upper half-clamp defines a deformable wall which can be crimped to secure the sleeve nut.

16. A transverse connector for connecting generally parallel first and second spine rods to one another, the assembly comprising:
a transverse rod having first and second ends;
a first screw clamp assembly comprising a first anchor screw, a first sleeve nut, a first lower half-clamp, and a first upper half-clamp wherein the half-clamps of the first screw clamp assembly mate to define a first opening to receive the first spine rod and a second opening generally perpendicular to the first opening to receive the first end of the transverse rod; and
a second screw clamp assembly comprising a second anchor screw, a second sleeve nut, a second lower half-clamp, and a second upper half-clamp wherein the half-clamps of the second screw clamp assembly mate to define a third opening to receive the second spine rod and a fourth opening generally perpendicular to the third opening to receive the second end of the transverse rod.

17. The transverse connector of claim 16 wherein the first and second sleeve nuts have a stepped external diameter and the first and second upper half-clamps each define an aperture with a stepped diameter.

18. The transverse connector of claim 17 wherein the first sleeve nut is recessed within the first upper half-clamp and the second sleeve nut is recessed within the second upper half-clamp.

19. The transverse connector of claim 16 wherein each anchor screw further comprises a shoulder located between the ends of the anchor screw and wherein each screw clamp assembly further comprises a spacer to be placed around the anchor screw to be positioned between the bone and the shoulder.

20. The transverse connector of claim 16 wherein each upper half-clamp is crimped to secure the sleeve nut.
